(19) United States
(12) Patent Application Publication
(10) Pub. No.: US 2022/0138347 A1
(43) Pub. Date: May 5, 2022

(54) RISK EVALUATION OF TRANSMISSION OF PATHOGENS IN A SPACE

(71) Applicant: International Business Machines Corporation, Armonk, NY (US)

(72) Inventors: JAMES MCDONAGH, Frodsham (GB); CARLOS PENA MONFERRER, Warrington (GB); PAOLO FRACCARO, Warrington (GB); LAURA-JAYNE GARDINER, Wirral (GB); Lan Ngoc HOANG, Lymm (GB); Peter Yoxall, Warrington (GB)

(21) Appl. No.: 17/083,349

(22) Filed: Oct. 29, 2020

(51) Int. Cl. G06F 21/62; G16H 50/80; G16H 50/30; G16H 50/50; G16H 50/20; G16H 40/20; G06Q 10/10; G06Q 50/26; G06Q 50/16; G06N 3/08; A61B 5/00

(52) U.S. Cl. CPC G06F 21/6254; G16H 50/80; G16H 50/30; G16H 50/50; G16H 50/20; G16H 40/20; A61B 2560/0252; G06Q 50/265; G06Q 50/163; G06N 3/08; A61B 5/7275; A61B 2576/00; G06Q 10/10

(57) ABSTRACT

Evaluating certain risks that are involved with respect to the transmission of pathogens in a given physical space. Some aspects of the present invention include applying privacy-preserving artificial intelligence (AI) algorithms and physics-based simulations on image data to characterize the potential source of pathogens. A multi-outcome artificial intelligence (AI) model that identifies object interactions and human actions, combined with physics-based simulations is used to accurately evaluate pathogen load distributions and infection risks.

### BACKGROUND

The present invention relates to risk evaluation of transmission of pathogens in a space, and more specifically, to risk evaluation using image data.

Indirect transmission of infectious diseases is a serious public health threat. Respiratory infections can be transmitted indirectly through respiratory droplets when an infected person either coughs, sneezes, or touches a surface after having touched their mouth, nose, and/or eyes.

According to a growing body of scientific research, certain viruses can persist for several hours within the air and even longer on cardboard or solid surfaces. Studies have also shown that certain viruses can persist for days on materials such as plastic or stainless-steel viable. For example, measles can survive for hours on surfaces. This is also true for other more common infections such as the seasonal flu, common cold and norovirus.

### SUMMARY

According to an aspect of the present invention there is a computer-implemented method for risk evaluation of transmission of pathogens in a space, comprising: receiving analyzed image data of a space over a period of time identifying actions and characteristics of people in the space; modeling anonymized actions of people with characteristics in the space to provide predicted effects of the actions on identified areas in the space; modeling a physics-based simulation of a spread of a given type of pathogen in the area using the predicted effects of the actions and using ambient environment data of the area; and dynamically providing a risk estimate for the given type of pathogen for an area using the physics-based simulation for weighting risk levels of the actions in the areas.

According to another aspect of the present invention there is a system for risk evaluation of transmission of pathogens in a space, comprising: a processor and a memory configured to provide computer program instructions to the processor to execute the function of the component: an image analysis receiving component for receiving analyzed image data of a space over a period of time identifying actions and characteristics of people in the space; an action modeling component for modeling anonymized actions of people with characteristics in the space to provide predicted effects of the actions on identified areas in the space; a physics-based modeling component for modeling a physics-based simulation of a spread of a given type of pathogen in the area using the predicted effects of the actions and using ambient environment data of the area; and a dynamic risk component for dynamically providing a risk estimate for the given type of pathogen for an area using the physics-based simulation for weighting risk levels of the actions in the areas.

According to a further aspect of the present invention there is a computer program product for risk evaluation of transmission of pathogens in a space, the computer program product comprising a computer readable storage medium having program instructions embodied therewith, the program instructions executable by a processor to cause the processor to: analyze image data of a space over a period of time to identify actions and characteristics of people in the space; model anonymized actions of people with characteristics in the space to provide predicted effects of the actions on identified areas in the space; model a physics-based simulation of a spread of a given type of pathogen in the area using the predicted effects of the actions and using ambient environment data of the area; and dynamically provide a risk estimate for the given type of pathogen for an area using the physics-based simulation for weighting risk levels of the actions in the areas.

The computer readable storage medium may be a non-transitory computer readable storage medium and the computer readable program code may be executable by a processing circuit.

### BRIEF DESCRIPTION OF THE DRAWINGS

The subject matter regarded as the invention is particularly pointed out and distinctly claimed in the concluding portion of the specification. The invention, both as to organization and method of operation, together with objects, features, and advantages thereof, may best be understood by reference to the following detailed description when read with the accompanying drawings.

FIG. 1 is a flow chart of an example embodiment of a method in accordance with the present invention;

FIG. 2 is a schematic diagram of an example embodiment of a method with reference to system components in accordance with the present invention;

FIGS. 3A, 3B, 3C, 3D, 3E, 3F and 3G are schematic diagrams illustrating the processing of image data used in aspects of the methods in accordance with the present invention;

FIG. 4A is block diagram of an example embodiment of a system in accordance with the present invention;

FIG. 4B is a block diagram of an example embodiment of a system in accordance with the present invention;

FIG. 5 is a block diagram of a computer system or cloud server in which embodiments of the present invention may be implemented;

FIG. 6 is a schematic diagram of a cloud computing environment in which embodiments of the present invention may be implemented; and

FIG. 7 is a diagram of abstraction model layers of a cloud computing environment in which embodiments of the present invention may be implemented.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numbers may be repeated among the figures to indicate corresponding or analogous features.

### DETAILED DESCRIPTION

A method and system is provided for targeted public space sanitization by applying privacy-preserving artificial intelligence (AI) algorithms and physics-based simulations on video footage. Video footage from sources such as closed-circuit television (CCTV) is used to characterize the potential source of pathogens, such as viruses or bacteria, transmitted indirectly by people in order to predict pathogen load over space and time and to obtain dynamic risk assessments to optimally sanitize public spaces.

Anonymized video data is used to characterize the risk of an individual transferring infectious matter to specific areas or objects. A multi-outcome AI model, which identifies object interactions and people's actions, combined with physics-based simulations is used to accurately evaluate pathogen load distributions and infection risks. A dynamic risk function is then used to consume predicted variables from the computational models accounting for material properties, human actions, microbiology, and physics to provide a heuristic for infection risk. This enables sanitization actions to be derived and automatically executed from the video data.

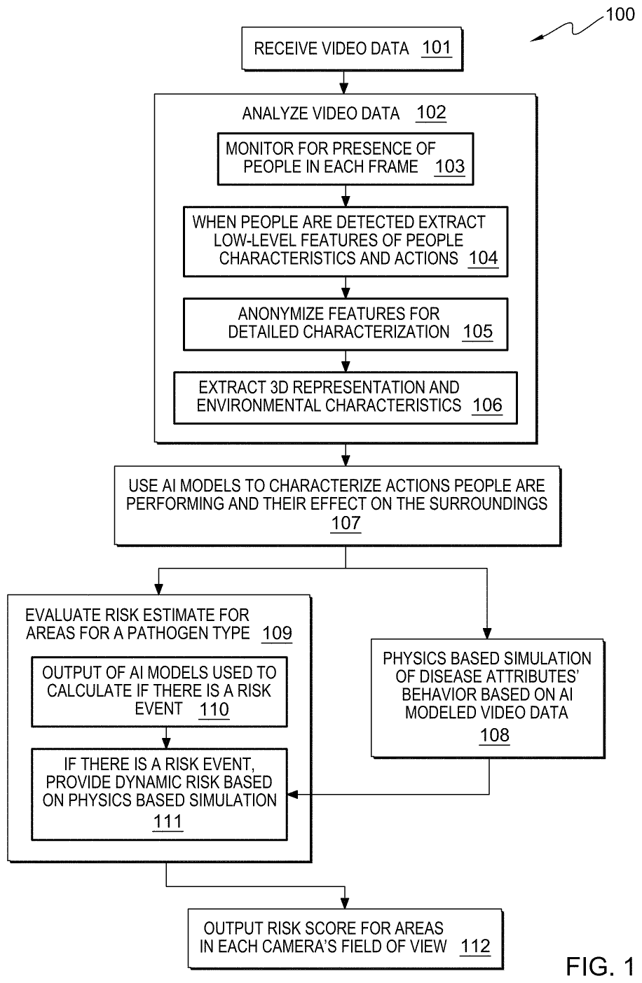

Referring to FIG. 1, a flow chart 100 shows and example embodiment of the described method for automated risk evaluation of transmission of pathogens in a space from video data.

The method receives 101 video data in the form of a series of video images of a space and analyzes 102 the image data of the space over a period of time to identify actions and characteristics of people in the space.

Analyzing 102 the image data includes monitoring 103 for people in each frame and extracting 104 low-level features of peoples' actions and characteristics to represent boundary conditions for modeling. Analyzing 102 the image data also includes anonymizing 105 features of people to identify characterizations such as poses and edges of a person while maintaining privacy. An intelligent method of filtering information via data extraction may be used so that the ongoing stream only contains the height and location of the source, rather than the sensitive individual data.

Analyzing 102 the image data may also include extracting 106 a three-dimensional representation of the space and its environmental characteristics. Additional inputs may also be received regarding the space such as environmental information including temperature, humidity, air flow, etc. and information regarding the surface materials of surfaces in the space including fixed surfaces such as floors, wall, and shelving, and variable surfaces such as objects on shelves and other moveable objects such as equipment.

The analyzed image data is received at a modeling system and the method uses AI modeling 107 to interpret the output of the video data analysis. The modeling anonymizes actions of people with characteristics in the space to provide predicted effects of the actions on identified areas and objects in the space. Predicted effects of the actions on identified areas in the space may include predicted effects in air flow and on surfaces in the area. The anonymization removes need to detect personal feature by basing the modeling on characteristics of people. The modeling includes applying a model for detecting actions or people that emit particles, for example, coughing, breathing, sneezing, touching surfaces, etc., and includes applying a model for effects of actions based on characteristics of people that may be trained with subjects of different characteristics for different actions, for example, a large person sneezing, or a running person breathing heavily.

Vision recognition and modeling systems may be used that integrate both objects and actions at the same time. Object detection may detect objects such as people, and actions may be labeled such as talking, walking, or touching. This integration of objects and actions is necessary to identify the cause and the consequence of actions in detected situations and to contextualize the risks. Simultaneously detecting of the actions and the objects causing and receiving the actions may highlight that in one case a touched object is plastic while another object is metal, which require different temporal attenuation of viral load.

The method also uses modeling of a physics-based simulation 108 of a spread of a given type of pathogen in the area using the predicted effects of the actions and using ambient environment data of the area. Modeling a physics-based simulation includes using a three-dimensional representation of the space and environmental characteristics for modeling particle dispersion in the space and for biological modeling of evolution of pathogen load.

Modeling a physics-based simulation may use a computation model of direct contact pathogen spread and a computation model for fluid flow for pathogen dispersal including a biological model for pathogen load over time. This may include inputting lifetime data indicative of longevity of pathogens on materials of surfaces in the space and ambient data indicative of ambient climate, airflows in the space, and data indicative of trajectories of airborne pathogens released through actions of people.

A risk estimate function is used to evaluate 109 risk for a type of pathogen in areas of the space. The risk estimate function consumes predicted variables from the computational models.

An initial risk evaluation may be carried out 110 based on outputs of the AI models of peoples' actions in the areas to determine if there is a risk event. This may include initially applying weightings for a type of action in combination with pathogen attributes for the action. Pathogen attributes may be transmission rate, prevalence in population, interaction with action and objects, etc. Therefore, there may be risk evaluations evaluated for multiple different pathogens or types of pathogens to build a combined risk evaluation.

If a risk event is determined, then additional evaluation may be carried out based on physics-based simulation. The initial risk evaluation may be determined very quickly and may enable a risk event to be acted on immediately if an urgent situation is found, even without the additional dynamic scoring based on physics-based simulation.

A dynamic risk evaluation may be carried out 111 if there is a determined risk event for the given type of pathogen using the physics-based simulation for weighting risk levels of the actions in the areas including dynamically updating the weightings based on projected pathogen attribute behavior. The physics-based simulation may be carried out with N models for N pathogens or grouped types of pathogens as the pathogen attribute behavior may vary for different pathogens.

Dynamically providing the risk estimate may determine a risk estimate function for pixels in an image frame of the image data based on a decay rate of viable pathogen matter over an effect matrix of pixels.

The method may output 112 a risk score estimate for areas in each camera's field of view and this may be used for generating a sanitization plan automatically for the space as a function of the risk estimate for the areas. The method may provide a continuous monitor which discretises a space and ranks the risk in each discrete space to alert when cleaning or other actions are needed.

Dynamic evidence-based risk calculation and sanitization provide resource efficient cleaning and a greener operating model. This may be used in any places where sanitization is key, such as communal areas of hospitals, doctors' practices, care homes, or hotels.

The described method predicts pathogen load distribution from video footage for taking sanitization actions while preserving people's privacy. For instance, there is currently no effective tool which can evaluate the risk and assist in deciding when to switch from spot cleaning (for example, one shelf) to area-based cleaning (for example, an aisle) or even disinfecting a whole store. This means that a potential transmission hotspot may be missed, or whole-store cleaning might be executed when not necessary, leading to wasteful overuse of cleaning materials, which are known to cause environmental damage.

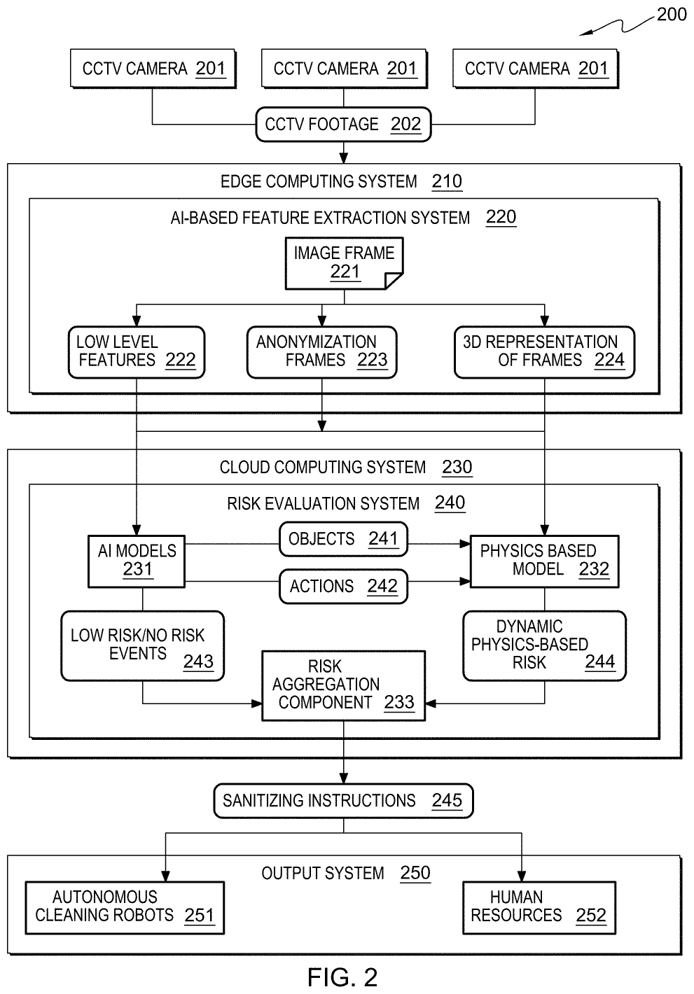

Referring to FIG. 2, a schematic diagram 200 shows the flow methods with reference to system components of the described system.

CCTV footage 202 or other forms of video data may be gathered from CCTV cameras 201 or other forms of camera that monitor a space, such as a public space, a shop, etc.

An edge computing system 210 may provide computing at the edge of the network for real time processing of image data. An AI-based feature extraction system 220 may be provided at the edge computing system 210 to extract features from received images 221, such as frames from the video data. The extraction may include extracting low-level features 222, anonymizing frames 223 of the image data, and providing 3D representation 224 of the frames.

The system may constantly process video footage (e.g. from CCTV cameras) on the edge to determine if a person is present in the video frames and to extract anonymized low-level features of a person (or multiple people) and ambient details from the footage.

On the edge, CCTV footage from each camera is constantly processed to monitor the presence of people in each frame. If people are detected, the following features and additional information are extracted for further processing on a cloud computing system 230.

The characteristics of droplets released by humans depend on both actions and physiology of the subjects. Therefore, information in the form of low-level features 222 is extracted that represents the boundary conditions for computing the dynamics of the droplets and the carrying of pathogen load, such as viral load. Impulse, drop size (such as mean and deviation of a log or normal distribution), emission rate, and an estimated initial viral load are needed by the described physics-based simulations. In order to extract this information from the images and still preserve the identity of people in public spaces, a two-step process is performed.

In a first step, a multi convolutional neural network (CNN) model is used. A first model may be used for detecting the following list of sub-actions: vigorous breathing, low speaking, intermediate speaking, loud speaking, sneezing, and coughing. Characteristics of people such as age, gender, height, and weight are needed to complete the drop characterization. Instead of detecting these personal features, a second CNN is used for directly providing information such as forced vital capacity, mean drop size, deviation, and number density. This is achieved by training the network with labelled data on images with subjects of different characteristics for the different actions. This labelled data can be assigned from empirical correlations or synthetic data as required. In this way, a second step consists of directly inferencing these parameters from the anonymized image features.

Some of the actions described above might require a more detailed and computationally intensive characterization on the cloud (such as a human/object(s) interaction). To allow this, while preserving people's privacy, AI models are applied to anonymize features of people (that is, an image of the person's outline and pose are extracted) rather than focusing on any detail, such as a person's face. The anonymized frames 223 are then sent to the cloud and processed. This is possible by using a combination of convolutional neural networks, which identify different poses (such as person reaching a surface or a shelf), static objects (such as products) and edges of a person (such as his or her hands).

In order to run the physics-based simulations, a 3D representation 224 and environmental characteristics are needed. This may consist of a set of surfaces representing objects and external walls as well as inputs defining heating or air conditioning systems. Internal flows, humidity, and temperature will be estimated from these parameters. In those spaces where objects are expected to be moved or replaced an update will be performed based on the CCTV footage.

These low-level features 222, anonymized frames 223, and 3D representations 224 are sent to a cloud computing system 230 for immediate AI model processing 231 and physics-based simulation 232 to provide a dynamic risk characterization.

In a cloud computing system 230, a risk evaluation system 240 uses the low-level features 222 and anonymized frames 223 by different AI models 231 to characterize the actions 242 that people are performing and the affected objects 241 in areas in the space. This can range from picking up items and putting them back to identifying the estimated trajectory of respiratory droplets when people cough or sneeze. These are achieved using AI models 231 containing variations of long short-term memory (LSTM) and CNN components to do multi-head detection of objects and actions. Ultimately, the AI models extract information on contact area, contact duration, object material, trajectory path, velocity and acceleration of the head in the case of coughing or sneezing. This will help in detecting the objects at the receiving end of the actions and help delineate the risk zone based on the extent of individual objects.

The output of the AI models 231 is used to determine low risk or no risk events 243 that do not need further risk assessment, whilst other higher risk events are aggregated with the dynamic physics-based risk 244 obtained from a physics-based model 232. The physics-based model 232 receives the objects 241 and actions 242 modeled by the AI models 231 and applies physical and biological constraints to determine the distribution and load of the pathogens due to the peoples' actions 242 in relation to the objects 241. A risk aggregation component 233 determines a risk level of different areas of the space captured in the video frames.

Integration of AI and physics-based simulation makes it possible for dynamic, computationally efficient and evidence-based risk information to be collected. On the one hand, when people are detected, a network of AI models uses the approaches described above (i.e. anonymization, object/action detection, and characterization) to provide inputs for risk score estimation and refinement via physics-based simulations that generate a dynamic risk. On the other hand, when people are not detected there is no need to transfer any data for further computation, thus reducing bandwidth usage. Furthermore, the proposed physics-based model not only includes state-of-the-art dispersion models, but accounts for coupled biological models representing the evolution of pathogen load. In addition to the data passed to the simulation as boundary conditions, the biological models remain interconnected with the AI models through source terms included in the equations for providing a combined simulation system.

Different embodiments of the described method and system may be provided through a variety of coupled physical simulation methods and AI models.

The output from the AI models 231 is used to calculate an initial risk event 243 to be sent back to the local requester. This can be done by using a multi-termed equation that, for each pixel in the frame, accounts for all actions of interest (e.g. coughing, sneezing, putting back an item) and their importance to the infection in the form of weights. An example is shown in Equation 1 below:

Risk(τ,AoE_{N},δ)=(RAe^{-δτAoE}^{_{A}}+RBe^{-δτAoE}^{_{B}}+RCe^{-δτAoE}^{_{C}}+ . . . RNe^{-δτAoE}^{_{N}})PM+a Equation 1

In Equation 1, the risk score RX is a weight associated with the relative importance to infection of an action (i.e. taking an item is less concerning than taking an item and putting it back). AoE_{Z} is an area of effect matrix effectively blurring the action effect over a number of pixels. PM is a pixel map of the image storing the risk value for a pixel and sigma (σ) is a noise term. This equation is re-evaluated over time (τ) applying a decay rate (δ) for viable pathogen matter.

Initially, the weights in the equation are set up heuristically to account for individual disease attributes such as disease transmission rate and prevalence in the population as well as the relative risk importance that different actions and human/objects interactions have, with affected objects material that is also taken into account (i.e. viral decay on different surfaces).

The weights are then dynamically updated based on projected pathogen distributions, affected areas, and decay information by the physics-based simulation, which takes as input all outputs from AI models on the edge and on the cloud. Briefly, these inputs include low-level anonymized features extracted from original frame, 3D representation of the scene, as well as detailed boundary conditions of people's actions, objects (e.g. material, location) and the interaction between the two. As part of the physics-based simulation 232, two sets of computational models are used.

In a first model, transfer of pathogens onto surfaces by direct contact is modeled. It is based on a model accounting for material properties of the objects involved, and the pathogens' mechanical characteristics. Based on adhesion and cohesion theory, contact area and duration the potential pathogen load transferred between objects will be computed.

In a second model, drops and pathogen load dynamics are modeled. It includes a multi-physics model for computing fluid flow, drop tracking, evaporation, and viral load. The fluid flow is computed with a CFD (computational fluid dynamics) code, while the drops are represented as a dispersed phase in the main air continuum medium. As a result of drop and flow characteristics the drop will evolve evaporating and remaining airborne droplet nuclei's or virions deposited on surfaces. A biological model is associated with the drops for computing the viral load as it evolves over time based on virus properties and variables such as local temperature and humidity values. Remaining droplet nuclei's or virions are included in the calculation until they are considered deactivated

The output from the physics-based models 232 is then integrated with the initial risk 243 calculated based on the AI models 231 output. Ultimately, for each camera a risk score is produced for each area in the shot, allowing staff or autonomous robots to prioritize their sanitation operations 245. For example, the system may allow areas to be identified that require immediate attention (e.g. areas where people coughed) and other areas to be disregarded where no risk was detected.

The results may be combined to form an evidence-based risk score and returned to the location, allowing local sanitization resources to be deployed where most necessary. This may be used by an output system 250 for controlling autonomous cleaning robots 251 and/or making recommendations to human resources 252.

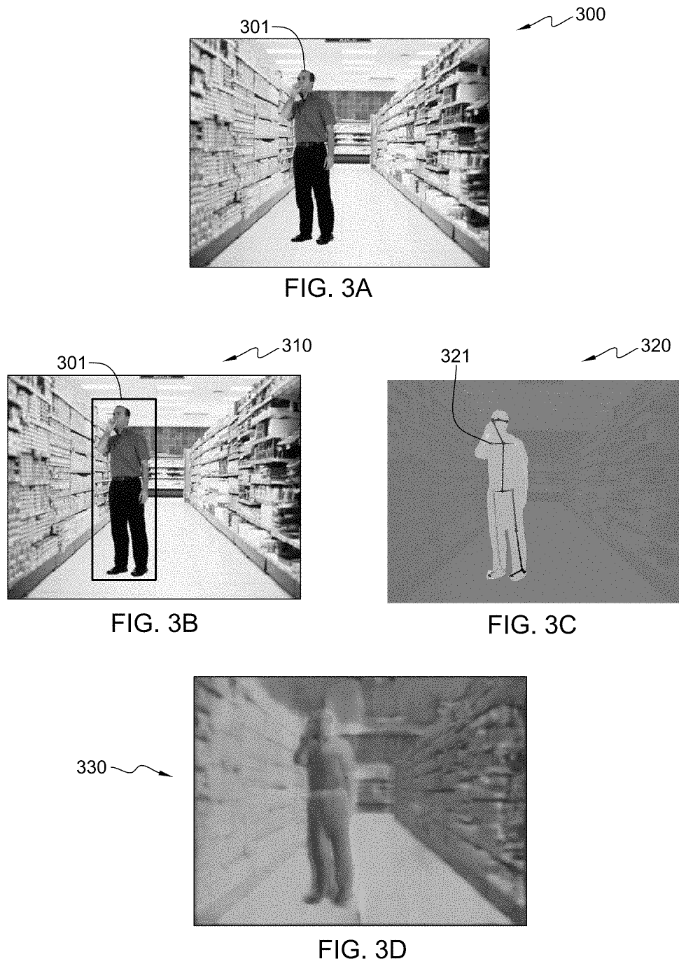

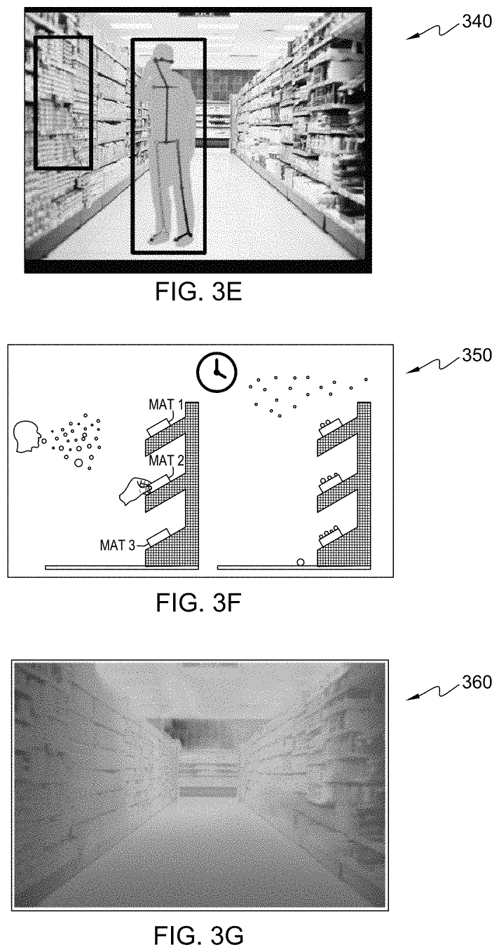

Referring to FIGS. 3A, 3B, 3C and 3D, example image frames are shown to illustrate the image processing carried out. This processing may be carried out at the AI-based feature extraction system 220 of the edge computing system 210. FIG. 3A shows a captured image frame 300 including a person 301 standing in a shop looking at objects on shelves and coughing. FIG. 3B shows the captured image frame 310 with low-level feature extraction of a person shown by a surrounding rectangle 311. FIG. 3C shows the captured image frame 320 with an anonymization area for the person by showing lines 321 to identify different poses. FIG. 3D shows the extraction of a 3D representation 330 of the space of the image, in this case, the form of the shelves containing objects and the floor.

Referring to FIGS. 3E, 3F and 3G, example image frames are shown to illustrate the modeling carried out. This modeling may be carried out at the risk evaluation system 240 at the cloud computing system 230. FIG. 3E shows an AI modeling of the image 340 to determine if there is a risk event based on the objects and actions. In this example, the person is coughing and an area of objects on the selves adjacent the person is identified as being affected by the action of the person coughing. FIG. 3F shows a physics-based simulation of the space 350 captured by the image showing particle dispersion and pathogen load on objects, which vary over time. In this example, the effect of the person coughing is modeled as dispersing particles towards the objects on the shelves. FIG. 3G shows a visualization of the risk aggregation 360 with darker areas showing a higher level of risk. In practice, such a visualization may provide a color representation of areas of high pathogen load.

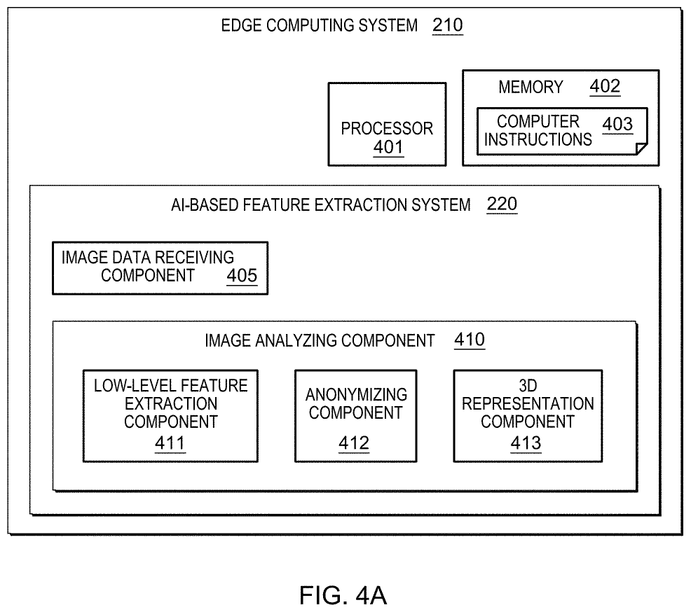

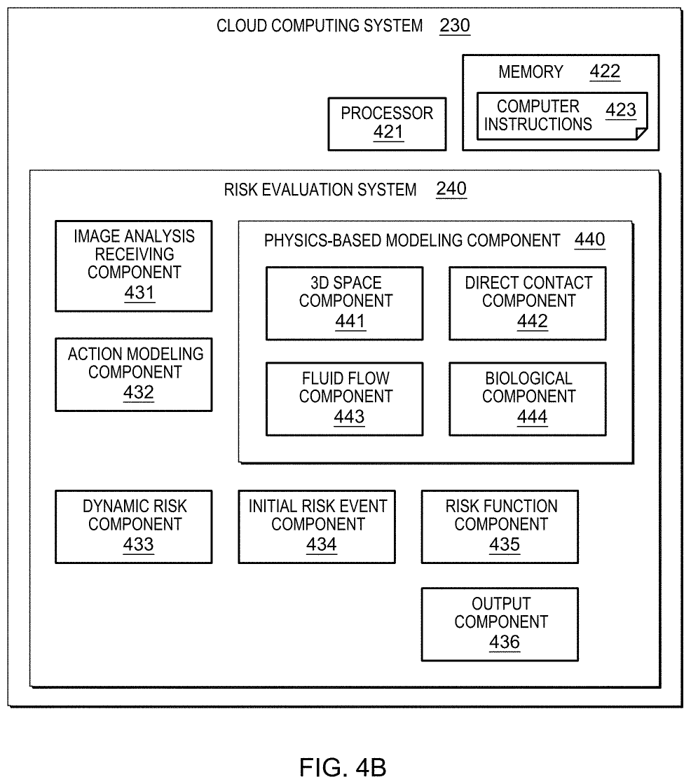

Referring to FIG. 4A, a block diagram shows an edge computing system 210 providing an AI-based feature extraction system 220. Referring to FIG. 4B, a block diagram shows a cloud computing system 230 providing an example embodiment of a risk evaluation system 240. In other embodiments, the AI-feature extraction system 220 and the risk evaluation system 240 may be provided at a same computing system.

The edge computing system 210 may be provided in proximity to the source of video data for real time processing and may include at least one processor 401, a hardware module, or a circuit for executing the functions of the described components which may be software units executing on the at least one processor. Multiple processors running parallel processing threads may be provided enabling parallel processing of some or all of the functions of the components. Memory 402 may be configured to provide computer instructions 403 to the at least one processor 401 to carry out the functionality of the components.

The AI-based feature extraction system 220 may include an image data receiving component 405 and an image analyzing component 405 for analyzing image data received from video camera recording a space over a period of time to identify actions and characteristics of people in the space. The image analyzing component 410 may include a low-level feature extraction component 411 for monitoring for people and extracting low-level features of peoples' actions and characteristics to represent boundary conditions for modeling and an anonymizing component 412 for anonymizing features of people to identify poses and edges of a person. The image analyzing component 410 may also include a 3D representation component 413 for providing a 3D representation of the space from the video data.

The cloud computing system 230 may be provided remotely to the source of video data and may include at least one processor 421, a hardware module, or a circuit for executing the functions of the described components which may be software units executing on the at least one processor. Multiple processors running parallel processing threads may be provided enabling parallel processing of some or all of the functions of the components. Memory 422 may be configured to provide computer instructions 423 to the at least one processor 421 to carry out the functionality of the components. In another embodiment, the risk evaluation system 240 may be provided on a non-cloud computing system locally to the edge computing system 210 or at a connected location.

The risk evaluation system 240 may include an image analysis receiving component 431 for receiving analyzed image data of a space over a period of time identifying actions and characteristics of people in the space and an action modeling component 432 for modeling anonymized actions of people with characteristics in the space to provide predicted effects of the actions on identified areas in the space.

The risk evaluation system 240 may include a physics-based modeling component 440 for modeling a physics-based simulation of a spread of a given type of pathogen in the area using the predicted effects of the actions and using ambient environment data of the area. The physics-based modeling component 440 may include: a three-dimensional space component 441 for using a three-dimensional representation of the space and environmental characteristics for modeling particle dispersion in the space; a direct contact component 442 for modeling of direct contact pathogen spread in the space; a fluid flow component 443 for modeling of fluid flow for pathogen dispersal in the space; and a biological component 444 for modeling pathogen behavior over time in the space.

The risk evaluation system 240 may include a dynamic risk component 433 for dynamically providing a risk estimate for the given type of pathogen for an area using the physics-based simulation for weighting risk levels of the actions in the areas and an initial risk event component 434 for modeling anonymized actions of people to determine if there is a risk event and, if so, the dynamic risk component models a physics-based simulation is used to dynamically evaluate the risk event.

The risk evaluation system 240 may include a risk function component 435 for evaluating a risk function using weighting risk levels of the actions in the areas including initially applying weightings for a type of action in combination with pathogen attributes for the action and dynamically updating the weightings based on projected pathogen attribute behavior based on physics-based simulation. The risk function component 435 mat include determining a risk estimate function for pixels in an image frame of the image data based on a decay rate of viable pathogen matter over an effect matrix of pixels.

The risk evaluation system 240 may include an output component 436 for generating a sanitization plan automatically for the space as a function of the risk estimate for the areas.

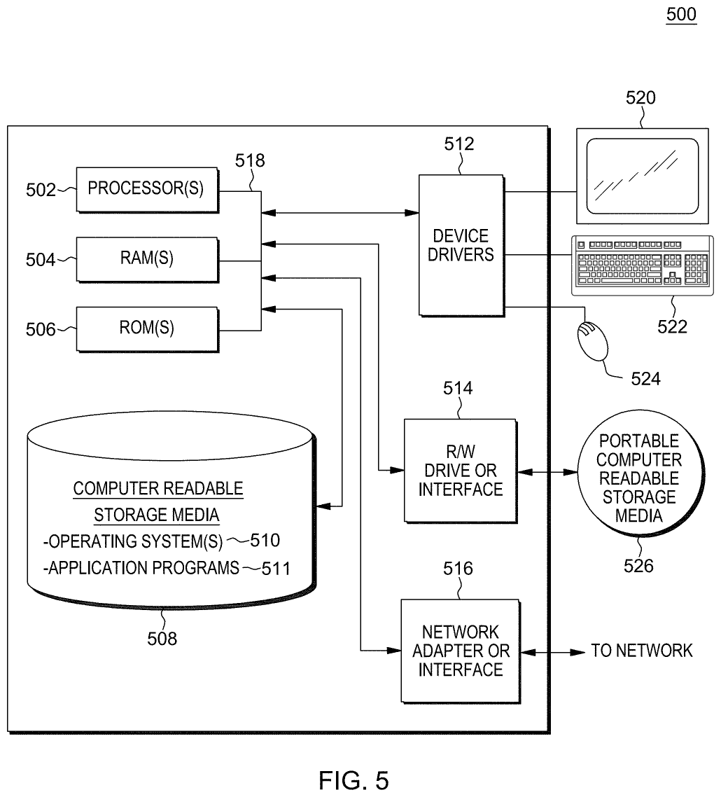

FIG. 5 depicts a block diagram of components of a computing system 500 as used for the edge computing system 210 and the cloud computing system 230, in accordance with an embodiment of the present invention. It should be appreciated that FIG. 5 provides only an illustration of one implementation and does not imply any limitations with regard to the environments in which different embodiments may be implemented. Many modifications to the depicted environment may be made.

The computing system can include one or more processors 502, one or more computer-readable RAMs 504, one or more computer-readable ROMs 506, one or more computer readable storage media 508, device drivers 512, read/write drive or interface 514, and network adapter or interface 516, all interconnected over a communications fabric 518. Communications fabric 518 can be implemented with any architecture designed for passing data and/or control information between processors (such as microprocessors, communications and network processors, etc.), system memory, peripheral devices, and any other hardware components within the system.

One or more operating systems 510, and application programs 511, such as the AI-based feature extraction system 220 and the risk evaluation system 240 are stored on one or more of the computer readable storage media 508 for execution by one or more of the processors 502 via one or more of the respective RAMs 504 (which typically include cache memory). In the illustrated embodiment, each of the computer readable storage media 508 can be a magnetic disk storage device of an internal hard drive, CD-ROM, DVD, memory stick, magnetic tape, magnetic disk, optical disk, a semiconductor storage device such as RAM, ROM, EPROM, flash memory, or any other computer readable storage media that can store a computer program and digital information, in accordance with embodiments of the invention.

The computing system can also include a R/W drive or interface 514 to read from and write to one or more portable computer readable storage media 526. Application programs 511 on the computing system can be stored on one or more of the portable computer readable storage media 526, read via the respective R/W drive or interface 514 and loaded into the respective computer readable storage media 508.

The computing system can also include a network adapter or interface 516, such as a TCP/IP adapter card or wireless communication adapter. Application programs 511 on the computing system can be downloaded to the computing device from an external computer or external storage device via a network (for example, the Internet, a local area network or other wide area networks or wireless networks) and network adapter or interface 516. From the network adapter or interface 516, the programs may be loaded into the computer readable storage media 508. The network may comprise copper wires, optical fibers, wireless transmission, routers, firewalls, switches, gateway computers and edge servers.

The computing system can also include a display screen 520, a keyboard or keypad 522, and a computer mouse or touchpad 524. Device drivers 512 interface to display screen 520 for imaging, to keyboard or keypad 522, to computer mouse or touchpad 524, and/or to display screen 520 for pressure sensing of alphanumeric character entry and user selections. The device drivers 512, R/W drive or interface 514, and network adapter or interface 516 can comprise hardware and software stored in computer readable storage media 508 and/or ROM 506.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be accomplished as one step, executed concurrently, substantially concurrently, in a partially or wholly temporally overlapping manner, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Cloud Computing

It is to be understood that although this disclosure includes a detailed description on cloud computing, implementation of the teachings recited herein are not limited to a cloud computing environment. Rather, embodiments of the present invention are capable of being implemented in conjunction with any other type of computing environment now known or later developed.

Cloud computing is a model of service delivery for enabling convenient, on-demand network access to a shared pool of configurable computing resources (e.g., networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported, providing transparency for both the provider and consumer of the utilized service.

Service Models are as follows:

Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure that includes a network of interconnected nodes.

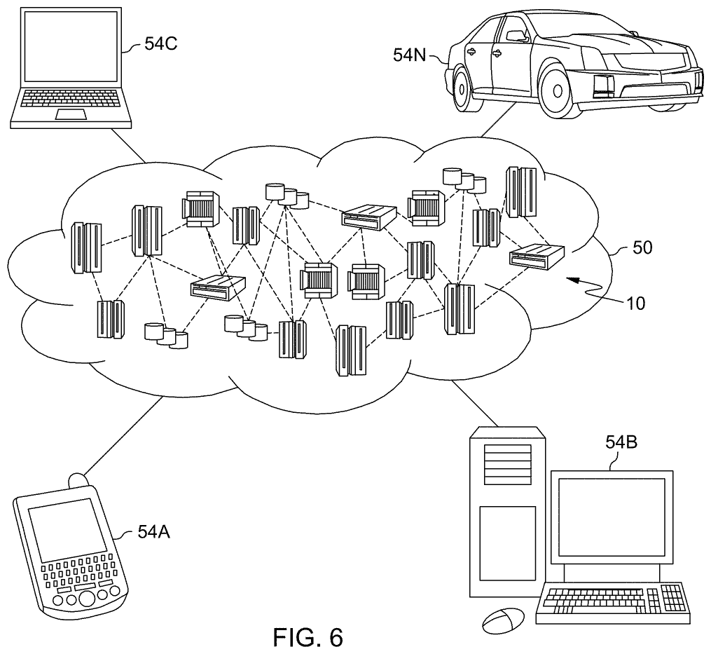

Referring now to FIG. 6, illustrative cloud computing environment 50 is depicted. As shown, cloud computing environment 50 includes one or more cloud computing nodes 10 with which local computing devices used by cloud consumers, such as, for example, personal digital assistant (PDA) or cellular telephone 54A, desktop computer 54B, laptop computer 54C, and/or automobile computer system 54N may communicate. Nodes 10 may communicate with one another. They may be grouped (not shown) physically or virtually, in one or more networks, such as Private, Community, Public, or Hybrid clouds as described hereinabove, or a combination thereof. This allows cloud computing environment 50 to offer infrastructure, platforms and/or software as services for which a cloud consumer does not need to maintain resources on a local computing device. It is understood that the types of computing devices 54A-N shown in FIG. 6 are intended to be illustrative only and that computing nodes 10 and cloud computing environment 50 can communicate with any type of computerized device over any type of network and/or network addressable connection (e.g., using a web browser).

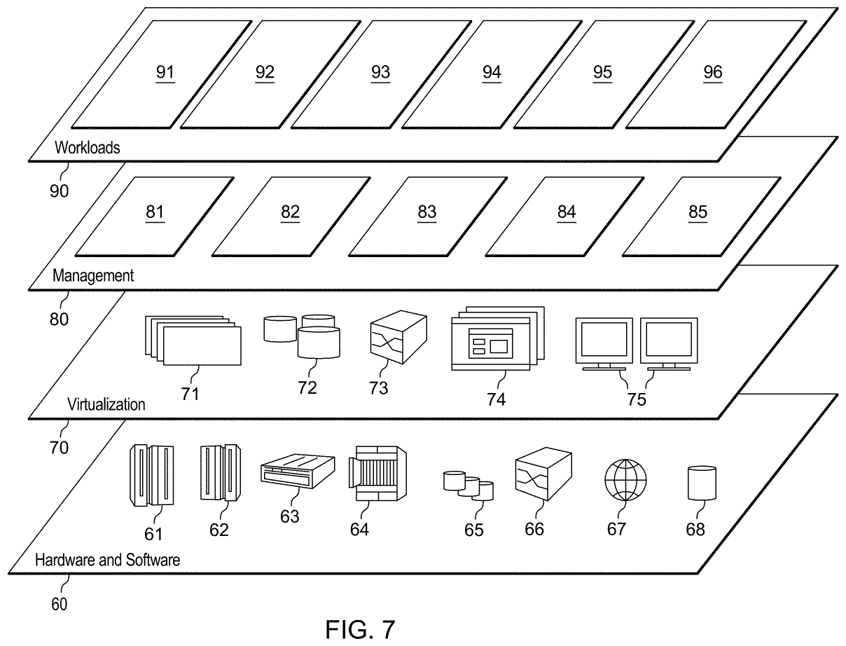

Referring now to FIG. 7, a set of functional abstraction layers provided by cloud computing environment 50 (FIG. 6) is shown. It should be understood in advance that the components, layers, and functions shown in FIG. 7 are intended to be illustrative only and embodiments of the invention are not limited thereto. As depicted, the following layers and corresponding functions are provided:

Hardware and software layer 60 includes hardware and software components. Examples of hardware components include: mainframes 61; RISC (Reduced Instruction Set Computer) architecture based servers 62; servers 63; blade servers 64; storage devices 65; and networks and networking components 66. In some embodiments, software components include network application server software 67 and database software 68.

Virtualization layer 70 provides an abstraction layer from which the following examples of virtual entities may be provided: virtual servers 71; virtual storage 72; virtual networks 73, including virtual private networks; virtual applications and operating systems 74; and virtual clients 75.

In one example, management layer 80 may provide the functions described below. Resource provisioning 81 provides dynamic procurement of computing resources and other resources that are utilized to perform tasks within the cloud computing environment. Metering and Pricing 82 provide cost tracking as resources are utilized within the cloud computing environment, and billing or invoicing for consumption of these resources. In one example, these resources may include application software licenses. Security provides identity verification for cloud consumers and tasks, as well as protection for data and other resources. User portal 83 provides access to the cloud computing environment for consumers and system administrators. Service level management 84 provides cloud computing resource allocation and management such that required service levels are met. Service Level Agreement (SLA) planning and fulfillment 85 provide pre-arrangement for, and procurement of, cloud computing resources for which a future requirement is anticipated in accordance with an SLA.

Workloads layer 90 provides examples of functionality for which the cloud computing environment may be utilized. Examples of workloads and functions which may be provided from this layer include: mapping and navigation 91; software development and lifecycle management 92; virtual classroom education delivery 93; data analytics processing 94; transaction processing 95; and image processing and risk evaluation processing 96.

A computer program product of the present invention comprises one or more computer readable hardware storage devices having computer readable program code stored therein, said program code executable by one or more processors to implement the methods of the present invention.

A computer system of the present invention comprises one or more processors, one or more memories, and one or more computer readable hardware storage devices, said one or more hardware storage device containing program code executable by the one or more processors via the one or more memories to implement the methods of the present invention.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

Improvements and modifications can be made to the foregoing without departing from the scope of the present invention.

What is claimed is:

1. A computer-implemented method for risk evaluation of transmission of pathogens in a space, comprising:
receiving analyzed image data of a space over a period of time identifying actions and characteristics of people in the space;
modeling anonymized actions of people with characteristics in the space to provide predicted effects of the actions on identified areas in the space;
modeling a physics-based simulation of a spread of a given type of pathogen in the area using the predicted effects of the actions and using ambient environment data of the area; and
dynamically providing a risk estimate for the given type of pathogen for an area using the physics-based simulation for weighting risk levels of the actions in the areas.

2. The method as claimed in claim 1, including generating a sanitization plan automatically for the space as a function of the risk estimate for the areas.

3. The method as claimed in claim 1, wherein modeling anonymized actions of people determines if there is a risk event and, if so, modeling a physics-based simulation is used to dynamically evaluate the risk event.

4. The method as claimed in claim 1, wherein weighting risk levels of the actions in the areas includes initially applying weightings for a type of action in combination with pathogen attributes for the action and dynamically updating the weightings based on projected pathogen attribute behavior based on physics-based simulation.

5. The method as claimed in claim 1, wherein dynamically providing a risk estimate includes determining a risk estimate function for pixels in an image frame of the image data based on a decay rate of viable pathogen matter over an effect matrix of pixels.

6. The method as claimed in claim 1, wherein predicted effects of the actions on identified areas in the space include predicted effects in air flow and on surfaces in the area.

7. The method as claimed in claim 1, wherein the analyzed image data includes extracted low-level features of peoples' actions and characteristics to represent boundary conditions for modeling.

8. The method as claimed in claim 1, wherein the analyzed image data includes anonymized features of people to identify poses and edges of a person.

9. The method as claimed in claim 1, wherein modeling a physics-based simulation includes using a three-dimensional representation of the space and environmental characteristics for modeling particle dispersion in the space and for biological modeling of evolution of pathogen load.

10. The method as claimed in claim 1, wherein modeling a physics-based simulation includes a computation model of direct contact pathogen spread and a computation model for fluid flow for pathogen dispersal including a biological model for pathogen load over time.

11. The method as claimed in claim 1, wherein modeling a physics-based simulation includes inputting lifetime data indicative of longevity of pathogens on materials of surfaces in the space and ambient data indicative of ambient climate, airflows in the space, and data indicative of trajectories of airborne pathogens released through actions of people.

12. The method as claimed in claim 1, wherein modeling a physics-based simulation of a spread of a given type of pathogen models the spread for multiple types of pathogen; and
dynamically providing a risk estimate determines a risk estimate based on multiple types of pathogen.

13. A computer system (CS) comprising:
a processor(s) set;
a machine readable storage device; and
computer code stored on the machine readable storage device, with the computer code including instructions and data for causing the processor(s) set to perform operations including the following:
an image analysis receiving component for receiving analyzed image data of a space over a period of time identifying actions and characteristics of people in the space,
an action modeling component for modeling anonymized actions of people with characteristics in the space to provide predicted effects of the actions on identified areas in the space,
a physics-based modeling component for modeling a physics-based simulation of a spread of a given type of pathogen in the area using the predicted effects of the actions and using ambient environment data of the area, and
a dynamic risk component for dynamically providing a risk estimate for the given type of pathogen for an area using the physics-based simulation for weighting risk levels of the actions in the areas.

14. The CS as claimed in claim 13, including a sanitization output component for generating a sanitization plan automatically for the space as a function of the risk estimate for the areas.

15. The CS as claimed in claim 13, including an initial risk event component for modeling anonymized actions of people to determine if there is a risk event and, if so, the dynamic risk component models a physics-based simulation is used to dynamically evaluate the risk event.

16. The CS as claimed in claim 13, including a risk function component for evaluating a risk function using weighting risk levels of the actions in the areas including initially applying weightings for a type of action in combination with pathogen attributes for the action and dynamically updating the weightings based on projected pathogen attribute behavior based on physics-based simulation.

17. The CS as claimed in claim 13, wherein the dynamic risk component includes determining a risk estimate function for pixels in an image frame of the image data based on a decay rate of viable pathogen matter over an effect matrix of pixels.

18. The CS as claimed in claim 13, including an image analyzing component for analyzing image data of a space over a period of time to identify actions and characteristics of people in the space and including a low-level feature extraction component for monitoring for people and extracting low-level features of peoples' actions and characteristics to represent boundary conditions for modeling.

19. The CS as claimed in claim 13, wherein the physics-based modeling component includes:
a three-dimensional space component for using a three-dimensional representation of the space and environmental characteristics for modeling particle dispersion in the space;
a direct contact component for modeling of direct contact pathogen spread in the space;
a fluid flow component for modeling of fluid flow for pathogen dispersal in the space; and
a biological component for modeling pathogen behavior over time in the space.

20. A computer program product (CPP) comprising:
a machine readable storage device; and
computer code stored on the machine readable storage device, with the computer code including instructions and data for causing a processor(s) set to perform operations including the following:
analyze image data of a space over a period of time to identify actions and characteristics of people in the space,
model anonymized actions of people with characteristics in the space to provide predicted effects of the actions on identified areas in the space,
model a physics-based simulation of a spread of a given type of pathogen in the area using the predicted effects of the actions and using ambient environment data of the area, and
dynamically provide a risk estimate for the given type of pathogen for an area using the physics-based simulation for weighting risk levels of the actions in the areas.
